(19) United States
(12) Patent Application Publication
(10) Pub. No.: US 2007/0141091 A1
(43) Pub. Date: Jun. 21, 2007

(54) BIGUANIDE OINTMENT AND METHOD OF TREATMENT AND PREVENTION OF INFECTIONS

(76) Inventors: Erning Xia, Penfield, NY (US); Hongna Wang, Fairport, NY (US); Praveen Tyle, Pittsford, NY (US)

Correspondence Address: Bausch & Lomb Incorporated, One Bausch & Lomb Place, Rochester, NY, 14604-2701, US

(21) Appl. No.: 11/610,893

(22) Filed: Dec. 14, 2006

Related U.S. Application Data
(60) Provisional application No. 60/752,455, filed on Dec. 21, 2005.
(60) Provisional application No. 60/760,510, filed on Jan. 20, 2006.
(60) Provisional application No. 60/760,880, filed on Jan. 20, 2006.
(60) Provisional application No. 60/782,478, filed on Mar. 15, 2006.
(60) Provisional application No. 60/830,319, filed on Jul. 12, 2006.
(60) Provisional application No. 60/830,326, filed on Jul. 12, 2006.

(51) Int. Cl. A61K 31/155; A61K 9/00

(52) U.S. Cl. 424/400; 514/635

(57) ABSTRACT

An ophthalmically acceptable composition comprising a biguanide antimicrobial agent and an ointment base. The invention further comprises administering the ophthalmically acceptable composition to the eye of a patient in need of treatment.

### CROSS REFERENCE TO RELATED APPLICATION

This application claims priority to U.S. Provisional Patent Application No.'s 60/752,455 filed Dec. 21, 2005; 60/760,510 filed Jan. 20, 2006; 60/760,880 filed Jan. 20, 2006; 60/782,478 filed Mar. 15, 2006; 60/830,319 filed Jul. 12, 2006 and 60/830,326 filed Jul. 12, 2006; the contents of each being incorporated by reference herein.

### BACKGROUND OF THE INVENTION

1. Field of the Invention

This invention relates to the treatment of topical infections including, ocular, otic, oral, vaginal, dermal and other topical infections.

2. Discussion of the Related Art

Ocular infections are not only uncomfortable conditions that require treatment, they often can result in permanent damage including corneal cysts and glaucoma, both of which can lead to a temporary or permanent loss of visual acuity and even blindness. Ocular infections include bacterial infections, viral infections, fungal infections and amoebal infections. These types of infections span four of the animal kingdoms and represent a wide range of genetic diversity.

Ocular infections include amoebal infections, fungal infections, viral infections and bacterial infections. These types of infections span four of the animal kingdoms and represent a wide range of genetic diversity.

An effective antimicrobial agent is one that is potent against a particular microbe yet is not toxic to human tissue. An antimicrobial agent that is potent against a single strain of microbe and has relatively little toxicity against human tissue is potentially valuable. An agent that is toxic against a wide range of antimicrobial agents, yet has relatively little toxicity against human tissue is considerably more valuable.

For some time, biguanide antimicrobial agents have been used to preserve ophthalmic solutions and have been known for their relatively low toxicity in patients compared to their preservative efficacy and other antimicrobial agents such as benkalkonium chloride. Biguanide antimicrobial agents include poly hexamethylene biguanide, chlorhexidine and alexidine.

To effectively preserve an ophthalmic composition, enough of the preservative is needed to prevent growth of S. Aureus, P. Aeruginosa and E. Coli bacteria and C. Albicans and A. Niger fungi over the shelf life of the product. Typically, a product will contain about the lowest amount of a preservative required to accomplish the desired effect. Between about 0.5 ppm and 1.5 ppm of a biguanide is needed to preserve most ophthalmic solutions.

Biguanide antimicrobial agents have been used as disinfectant solutions for contact lenses. To be considered a disinfectant, a solution needs sufficient antimicrobial agent to kill S. Aureus, P. Aeruginosa and S. Marcescens bacteria and C. Albicans and F. Solani fungi over the shelf life of the product. Furthermore, the solution must show efficacy in disinfecting contact lenses using the disinfecting regimen that is recommended on the product. For example one regimen may be to rinse the contact lenses in the solution, soak the contact lenses in the solution for six hours and rinse the contact lens in the solution again.

Disinfecting solutions containing antimicrobial agents include ReNu® Multiplus sold by Bausch & Lomb, Rochester, N.Y. ReNu® Multiplus is a multipurpose cleaning, conditioning and disinfecting solution for contact lenses that contains 3 ppm of polyhexamethylene biguanide. ReNu® with MoistureLoc is a multipurpose cleaning, conditioning and disinfecting solution for contact lenses that contains 3 ppm of alexidine.

Disinfecting solutions such as the one mentioned above are ophthalmically safe solutions. They are safe to administer to the eye of a patient. Contact lenses that have been rinsed with these solutions are placed in the eye. However, these solutions are not recommended for use as a medicament in the eye. There is no evidence to suggest that the level of antimicrobial agent in a multipurpose contact lens solution would be effective to treat ocular infection.

Several studies have been conducted on the effectiveness of polyhexamethylene biguanide and/or chlorhexidine for treatment of Acanthamoebal keratitis.

In Schuster, et al., “Opportunistic Amoebae: Challenges In Prophylaxis And Treatment,” Drug Resistance Updates: Reviews And Commentaries In Antimicrobial And Anticancer Chemotherapy, vol. 7(1) pp. 41-51 (Feb. 2004), Acanthamoeba keratitis, a non-opportunistic infection of the cornea, was found to respond well to treatment with chlorhexidine gluconate and polyhexamethylene biguanide, in combination with propamidine isothionate (Brolene), hexamidine (Desomodine), or neomycin.

In Rama et al., “Bilateral Acanthamoeba keratitis with late recurrence of the infection in a corneal graft: a case report,” European Journal of Ophthalmology, vol. 13 (3), pp. 311-4 (Apr. 2003), recurrences of Acanthamoeba keratitis in both eyes were successfully treated with a combination of hexamidine and neomycin, and with polyhexamethylene biguanide respectively.

Anita et al., “Role of 0.02% polyhexamethylene biguanide and 1% povidone iodine in experimental Aspergillus keratitis,” Cornea, Vol. 22 (2), pp. 138-41, (Ma. 2003) showed that polyhexamethylene biguanide (0.02%) is a moderately effective drug for experimental Aspergillus keratitis.

Sharma et al., “Patient characteristics, diagnosis and treatment of non-contact lens related Acanthamoeba keratitis,” British Journal of Ophthalmology, Vol. 84/10, pp. 1103-1108 (2000) illustrates the combination of polyhexamethylene biguanide and chlorhexidine.

Shelf life is an important issue for pharmaceuticals that treat ocular infection. Particularly, no less than 90% of an active agent can deteriorate over a two-year period of time to be approved by the Food and Drug Administration. Biguanides are somewhat unstable and degrade in an aqueous solution.

Thus, there is a need for a stable ophthalmic antimicrobial composition that is relatively non-toxic and effective against a wide range of microbes. The present invention addresses this and other needs.

### SUMMARY OF INVENTION

The present invention includes a composition for treating infectious disease. The composition comprises a biguanide antimicrobial agent in an amount effective to treat infectious disease and an ointment base. The present invention also includes a method of treating infectious disease. The method comprises administering an ophthalmically acceptable composition to the ocular region of a patient infected with an infectious disease. The ophthalmically acceptable composition comprises a biguanide antimicrobial agent and an ointment base. One benefit of medication in an ointment is a longer residence time in the eye.

In one embodiment, the ointment base is selected from the group consisting of petrolatums and ophthalmically compatible oils including mineral oil.

In one embodiment, the composition further comprises a surfactant. Typically the surfactant is selected from the group consisting of polysorbate, cremaphor, triton, poloxamine, poloxamer and tyloxapol.

In one embodiment, the biguanide antimicrobial agent is selected from the group consisting of polyhexamethylene biguanide, chlorhexidine and alexidine. Preferably, the biguanide antimicrobial agent is Alexidine.

In another embodiment, the biguanide is present in an amount ranging from 3 ppm to about 1.0 wt. % based upon the total amount the composition.

In yet another embodiment, the composition further comprises a penetration enhancer.

In still another embodiment, the penetration enhancer is present in an amount that is a minimum of about 0.01 wt. % and a maximum of about 5 wt. %.

In one embodiment, the stabilizer is in an amount effective to extend the shelf life a minimum of about 10%.

In another embodiment, the composition further comprises a viscosifier.

In yet another embodiment, the viscosifiers are selected from the group consisting of natural polysaccharides, natural gums, synthetic polymers, proteins and synthetic polypeptides that are capable of increasing viscosity and are ophthalmically acceptable.

In still another embodiment, the viscosifiers are selected from the group consisting of mucomimetics. Preferably, the viscosifier is a carboxyvinyl polymer in one embodiment.

In another embodiment, the method treats an infectious deasease that is a fungal infection.

In one embodiment, the method treats an infectious disease that is an amoebal infection, for example amoebal keratitis.

In another embodiment, the method treats an infectious disease that is a viral infection.

In one embodiment, the method treats an infectious disease that is a bacterial infection, for example bacterial conjunctivitis.

### DETAILED DESCRIPTION OF THE PREFERRED EMBODIMENT

The active ingredients are used in the topically administrable therapeutic compositions for microbial eye disease as well as other topical disease such as dermal or otic disease in accordance with the invention (although such compositions are occasionally referred to herein as “ophthalmic” and words of similar meaning, use of this abbreviation does not exclude the application of the invention in the nasal, otic, oral, vaginal or dermal fields).

The term “composition” as used herein, refers to various forms of the compounds or compositions of the present invention, including solids such as ointments, creams and gels.

The term “treating” refers to any indicia of success in the treatment or amelioration or prevention of an ocular disease, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an eye examination. Accordingly, the term “treating” includes the administration of the compounds or agents of the present invention to prevent or delay, to alleviate, or to arrest or inhibit development of the symptoms or conditions associated with disease. The term “therapeutic effect” refers to the reduction, elimination, or prevention of the disease, symptoms of the disease, or side effects of the disease in the subject. The composition of the invention herein is useful for the treatment of various medical conditions selected from the groups consisting of promoting wound healing, reduction of pathogens in open wounds, ocular disinfection or decontamination, antifungal therapy, ophthalmic, otology applications, treatment of viral infections, treatment of skin diseases, and tissue repair and regeneration, which method comprises using the composition of the invention by applying the composition to the site where treatment is required.

The compositions of the present invention possess activity toward microbes, i.e., antimicrobial activity. As used herein, the term “antimicrobial” is meant to include prevention, inhibition, termination, or reduction of virulence factor expression or function of a microbe. “Prevention” can be considered, for example, to be the obstruction or hindrance of any potential microbial growth. “Termination” can be considered, for example, to be actual killing of the microbes by the presence of the composition. “Inhibition” can be considered, for example, to be a reduction in microbial growth or inhibiting virulence factor expression or function of the microbe.

As used herein, “microbe” or “microbial agent” is meant to include any organism comprised of the phylogenetic domains bacteria and archaea, as well as unicellular and filamentous fungi (such as yeasts and molds), unicellular and filamentous algae, unicellular and multicellular parasites, and viruses that causes disease in a subject. Accordingly, such microbial agents include, but are not limited to, bacterial, viral, fungal, or protozoan pathogens.

Alexidine is a biguanide antimicrobial agent that is defined by the formula 1,1′-Hexamethylene-bis [5-(2-ethylhexyl)biguanide]. By biguanide antimicrobial agent it is meant an antimicrobial agent that has a biguanide substituent and have antimicrobial properties in an ophthalmically safe amount. Suitable biguanide antimicrobial agents include but are not limited to 1,1′-hexamethylene-bis[5-(p-chlorophenyl)biguanide] (Chlorhexidine) or water soluble salts thereof, 1,1′-hexamethylene-bis[5-(2-ethylhexyl)biguanide ] (Alexidine) or water-soluble salts thereof and poly(hexamethylene biguanide) (PHMB).

In one embodiment, the amount of antimicrobial agent in the ophthalmic composition is a minimum of about 1 ppm and a maximum of about 1 wt. %. Typically, the amount of antimicrobial agent in the ophthalmic ointment is a maximum of about 800 ppm, about 500 ppm, about 300 ppm, about 100 ppm, about 75 ppm, about 50 ppm, about 20 ppm, about 15 ppm or about 10 ppm.

In one embodiment, the ointment base is selected from the group consisting of petrolatums and ophthalmically compatible oils including mineral oil.

In one embodiment, the composition further comprises a surfactant. In another embodiment, the surfactant is selected from the group consisting of polysorbate, cremophor, triton, poloxamine, poloxamer and tyloxapol. In one embodiment, the surfactant is present in an amount that is a minimum of about 0.01 wt. % and a maximum of about 10 wt. %. Typically, the amount of surfactant is a minimum of about 2 wt. %, about 3 wt. %, about 4 wt. % or about 5 wt. % and/or a minimum of about 10 wt. %, about 9 wt. %, about 8 wt. %, about 7 wt. %, about 6 wt. % or about 5 wt. %.

In one embodiment, the ointment is a clear hydrophobic ointment with preferably no water present. In one embodiment, the amount of water is a maximum of about 10 wt. %, about 5 wt. %, about 3 wt. %, about 2 wt. % or about 1 wt. %.

In another embodiment, the ointment is an oil and water emulsion (i.e., a cream). Generally, the amount of water is a minimum of about 10 wt. % and a maximum of about 60 wt. %. Typically, the amount of water is a minimum of about 20 wt. %, about 30 wt. % and a maximum of about 55 wt. %, about 50 wt. %, about 45 wt. % and about 40 wt. %.

Due to the tendency of alexidine or other biguanide antimicrobial agents to hydrolyze in an aqueous solution it is desirable to include a stabilizer. A stabilizer is a compound that prevents the chemical degradation of an active agent in solution. Examples of stabilizers that are effective in an aqueous solution include but are not limited to hydroxyl alkyl phosphonate, Tetronics® 908, tyloxapol, cyclodextrin and derivatives, hyaluronic acid, sodium edetate, citric acid as well as other ophthalmically acceptable antioxidants, complexing agents and chelating agents and salts thereof. In one embodiment, preferred stabilizers are hydroxyalkyl phosphonate, ethylenediamine-tetraacetic acid, Tetronics® 908, tyloxapol, cyclodextrin or hyaluronic acid and ethylenediamine tetra acetic acid.

In one embodiment, the stabilizer is present in an amount effective to stabilize the compound. An amount effective to stabilize a compound means that the stabilizer is present in an amount that prevents deterioration of at least 90% of the compound in a period of 24 months. In another embodiment, the preferred stabilizer is present in a minimum amount of about 0.001 wt. %, about 0.005 wt. %, about 0.01 wt. % and/or a maximum amount of about 0.5 wt. %, about 0.3 wt. %, about 0.1 wt. %, about 0.08 wt. %, about 0.05 wt. %, about 0.03 wt. %, about 0.01 wt. %.

In another embodiment the effective shelf life of the antimicrobial agent is extended by a minimum of about 10 percent of the shelf life without the stabilizer. In another embodiment, the antimicrobial agent is extended by a minimum of about 20 percent, about 40 percent, about 80 percent, about 100 percent or about 200 percent.

Various anatomical barriers relating to the eye may underlie the poor intraocular penetrance of active ingredients. In this regard, the cornea is the principal barrier to entry of foreign substances. It has two distinct penetration barriers, the corneal epithelium and the corneal stroma. Thus, it is desirable to use a penetration enhancer to improve the penetration of the active ingredients of the present invention.

The penetration enhancer generally acts to make the cell membranes less rigid and therefore more amenable to allowing passage of drug molecules between cells. The penetration enhancers preferably exert their penetration enhancing effect immediately upon application to the eye and maintain this effect for a period of approximately five to ten minutes. The penetration enhancers and any metabolites thereof must also be non-toxic to ophthalmic tissues. One or more penetration enhancers will generally be utilized in a minimum amount of about 0.01 weight percent and/or a maximum of about 10 wt. %.

In one embodiment, the preferred penetration enhancers are benzalkonium chloride or ethylenediaminetetraacetic acid (EDTA). The preferred penetration enhancers of another embodiment are saccharide surfactants, such as dodecylmaltoside (“DDM”), and monoacyl phosphoglycerides, such as lysophosphatidylcholine. The saccharide surfactants and monoacyl phosphoglycerides, which may be utilized, as penetration enhancers in the present invention are known compounds. The use of such compounds to enhance the penetration of ophthalmic drugs is described in U.S. Pat. No. 5,221,696 the entire contents of which are incorporated by reference into the present specification. Penetration enhancers are present in an amount ranging from about 0.001 wt. % to about 3 wt. %.

Ointment compositions may likewise include a polyol co-solvent including but not limited to sorbitol, mannitol, glycerol, xylitol, propylene glycol and poly(ethylene glycol). In one embodiment, the polymeric cosolvent is preferably poly(ethylene glycol) and has a molecular weight that is a minimum of about 200 Da and a maximum of about 4000 Da. In another embodiment, the polymeric cosolvent is preferably propylene glycol or glycerol. In one embodiment, the polyol co-solvent is at a concentration that is a minimum of about 0.1 wt. % and a maximum of about 10 wt. %. Typically, the polyol cosolvent is at a concentration that is a minimum of about 0.5 wt. %, about 1 wt. %, about 2 wt. %, about 3 wt. %, about 4 wt. % or about 5 wt % and/or a maximum of about 9 wt. %, about 8 wt. %, about 7 wt. %, about 6 wt. % or about 5 wt. %.

The ointment composition optionally contains other pharmaceutical agents including anti-inflammatory agents, antiviral agents, antibacterial agents and antifungal agents. Anti-inflammatory agents include but are not limited to steroidal anti-inflammatory agents such as corticosteriods selected from the group consisting of cortisone, dexamethasone, fluorometholone, hydrocortisone, loteprednol, medrysone, methylprednisolone, prednisolone, prednisone, rimexolone, and triamcinolone and non-steroidal anti-inflammatory agents selected from the group consisting of cromolyn, diclofenac, flurbiprofen, ketorolac, lodoxamide, nedocromil, pemirolast, and suprofen. Antiviral agents include but are not limited to the agents selected from the group consisting of trifluorothymidine, ganciclovir, fomivirsen, vidarabine, cyclosporine, valganciclovir, amantadine, cidofovir, rabavirin, rimantadine, zanamivir, natamycin, flucytosine, griseofulvin and echinocandins. Antifungal agents include but are not limited to the agents selected from the group consisting of poluene antifungals, imidazole antifungals, triazole antifungals and allylamine antifungals. Antibacterial agents include but are not limited to the agents selected from the group consisting of bacitracin, chloramphenicol, ciprofloxacin, erythromycin, gatifloxacin, gentamicin, levofloxacin, moxifloxacin, ofloxacin, polymzin B, sulfonamides and tobramycin.

Specific Methods for Using the Compositions of the Invention

In one aspect, the compositions of the invention are administered or used topically.

The compounds of the current invention may be used to treat topical infections by incorporating them into creams, ointments or lotions for use in such conditions. Such creams, ointments or lotions might be used for a broad variety of skin conditions and may incorporate penetration enhancers in order to deliver the antimicrobial activity of the compound to microbes present beneath the outer (epidermis) layers of the skin.

Topical administration according to the present invention also includes the application of ointments and gels containing one or more biguanide antimicrobial agents to the eye or ear. The ointments and gels can include any substances known to the skilled composition chemist to be useful for the preparation of such ointments and gels.

Typically, the ointments and gels will include a base which permits diffusion of the drug into the tissue of the treated region. In exemplary embodiments of the present invention, the base will be comprised of white petrolatum and mineral oil and other substances known in the art as being appropriate for administration to the eye, e.g., anhydrous lanolin and/or polyethylene-mineral oil gel. The amount of a biguanide antimicrobial agent in the ointment or gel can vary widely depending on the type of composition, size of a unit dosage, kind of excipients, and other factors well known to those of ordinary skill in the art.

### EXAMPLES

Specific compositions are listed in the examples below:

### Example 1

### Ophthalmic Ointment of Alexidine—Composition 1

| Ingredients | % w/w |
|---|---|
| Alexidine | 500 ppm |
| White Petrolatum, U.S.P. | 80.00 |
| Propylene Glycol | 3.00 |
| Pluronic F127 | 1.00 |
| Mineral Oil | Qs to 100 |
| BAK | 0.10 |

### Example 2

### Ophthalmic Ointment of Alexidine—Composition 2

| Ingredients | % w/w |
|---|---|
| Alexidine | 200 ppm |
| White Petrolatum, U.S.P. | 50.00 |
| Propylene Glycol | 5.00 |
| Glycerin | 5.00 |
| Tween 20 | 2.00 |
| Vitamin E | 1.00 |
| BAK | 0.10 |
| Mineral Oil | Qs to 100 |

### Example 3

### Ophthalmic Ointment of Alexidine—Composition 3 (Preservative Free)

| Ingredients | % w/w |
|---|---|
| Alexidine | 200 ppm |
| Ciprofloxacin | 1.0 |
| White Petrolatum, U.S.P. | 50.00 |
| Glycerin | 5.00 |
| Pluronic F68 | 5.00 |
| BHT | 0.20 |
| Mineral Oil | Qs to 100 |

### Example 4

### Ophthalmic Ointment of Alexidine—Composition 4

| Ingredients | % w/w |
|---|---|
| Alexidine | 100 ppm |
| White Petrolatum, U.S.P. | 50.00 |
| Propylene Glycol | 5.00 |
| Glycerin | 5.00 |
| Triton 100 | 0.5 |
| EDTA | 1.00 |
| BAK | 0.50 |
| Mineral Oil | Qs to 100 |

### Example 5

### Otic Ointment of Alexidine

| Ingredients | % w/w |
|---|---|
| Alexidine | 0.05 |
| White Petrolatum, U.S.P. | 50.00 |
| Propylene Glycol | 5.00 |
| Glycerin | 5.00 |
| Tween 20 | 2.00 |
| Vitamin E | 1.00 |
| Methionine | 0.25 |
| Mineral Oil | Qs to 100 |

### Example 6

### HSV-1 Viral Suspension Assay

The Viral Suspension Assay was used to evaluate the antiviral properties of Alexidine against Herpes simplex virus type 1 when exposed in suspension for 1, 2, 5, and 10 minutes. The presence of virus (infectivity) was determined by monitoring the virus specific cytopathic effect (CPE) on an appropriate indicator cell line, rabbit kidney. Results are reported as Percent (%) Reduction in virus titer as compared to the corresponding virus control titer (Table 1). The titer of the virus controls were 7.5 log₁₀ following the one minute exposure time; 7.0 log₁₀ following the two minute exposure time; and 7.75 log₁₀ following both the five and ten minute exposure times. The results are listed in Table 1 and show that Alexidine at both 30 ppm and 99 ppm are effective agents against herpes simplex type-1 virus (HSV-1).

TABLE 1
Viral Suspension Assay Percent Reduction of Herpes simplex virus
type 1 after 1, 2, 5 and 10 Minute Exposure to Alexidine

| Alexidine | | | | |
|---|---|---|---|---|
| Test Concentration | 1 minute | 2 minutes | 5 minutes | 10 minutes |
| 30 ppm | 99.99% | 99.99% | 99.9994% | ≧99.99994% |
| 99 ppm | 99.999% | 99.994% | 99.9999% | ≧99.99994% |

### Example 7

### Adenovirus and Cytomegalovirus Testing

The Viral Suspension Assay was used to evaluate the antiviral properties of Alexidine against Adenovirus Type-4, Adenovirus Type-8 and Adenovirus Type-19 and Cytomegalovirus when exposed in suspension for 1, 2, 5, and 10 minutes. The presence of virus (infectivity) was determined by monitoring the virus specific cytopathic effect (CPE) on an appropriate indicator cell line, rabbit kidney. Results are reported as Percent (%) Reduction in virus titer as compared to the corresponding virus control titer (Table 1). The titer of the virus controls were 7.5 log₁₀ following the one minute exposure time; 7.0 log₁₀ following the two minute exposure time; and 7.75 log₁₀ following both the five and ten minute exposure times. The results are listed in Table 2 and show that Alexidine at both 30 ppm and 99 ppm are somewhat effective against viral strains of Adenovirus Type-4, Adenovirus Type-8, and Cytomegalovirus. However, Alexidine did not appear to be effective against the particular strain of Adenovirus Type-19 that was tested. Alexidine is a potent antimicrobial agent against Herpes Simplex-1 and has some effectiveness against certain strains of other viruses that cause ocular infection.

TABLE 2
Viral Suspension Assay Percent Reduction of Adenovirus Type-4,
Adenovirus Type-8 and Adenovirus Type-19 and Cytomegalovirus
after 1, 2, 5 and 10 Minute Exposure to Alexidine

| | Alexidine | Percent Reduction (%) | | | |
|---|---|---|---|---|---|
| | Test | 1 | 2 | 5 | 10 |
| Virus | Concentration | minute | minutes | minutes | minutes |
| Adenovirus | 30 ppm | 43.8 | — | 82.2 | 68.4 |
| type 4 | 99 ppm | 68.4 | — | 43.8 | 68.4 |
| Adenovirus | 30 ppm | 96.8 | 94.4 | 82.2 | 90.0 |
| type 8 | 99 ppm | 82.2 | 82.2 | 90.0 | 90.0 |
| Adenovirus | 30 ppm | No reduction | | | |
| type 19 | 99 ppm | | | | |
| Cytomegalovirus | 30 ppm | 43.8 | 68.4 | — | 43.8 |
| | 99 ppm | 98.2 | 99.0 | 99.8 | 99.98 |

It will be understood that the present invention is typically applied by administering a ribbon of ointment to the eye of a patient. In one embodiment, the ointment is placed in the conjuntival sac beneath the eye. Typically, the ointment is administered a minimum of once daily, two times daily or three times daily.

What is claimed is:

1. A method of treating infectious disease comprising administering an ophthalmically acceptable composition to the ocular region of a patient, the ophthalmically acceptable composition comprising a biguanide antimicrobial agent and an ointment base.

2. The method of claim 1, wherein the ointment base is selected from the group consisting of petrolatum and mineral oil.

3. The method of claim 1, wherein water, if any, is present in an amount that is a maximum of about 20 wt. %.

4. The method of claim 1, further comprising water in an amount that is a minimum of about 20 wt. % and a maximum of about 60 wt. %.

5. The method of claim 1, further comprising a surfactant.

6. The method of claim 1, wherein the surfactant is selected from the group consisting of polysorbate, cremophor, triton, poloxamine, poloxamer and tyloxapol.

7. The method of claim 1, wherein the surfactant is present in an amount ranging from about 0.01 wt. % to about 20 wt. %.

8. The method of claim 1, wherein the composition further comprises a penetration enhancer.

9. The method of claim 8, wherein the penetration enhancer is present in an amount that is a minimum of about 0.001 wt. % and a maximum of about 10 wt. %.

10. The method of claim 1, wherein the biguanide antimicrobial agent is Alexidine.

11. The method of claim 1, wherein the ophthalmically acceptable composition comprises a stabilizer is in an amount effective to extend the shelf life a minimum of about 10%.

12. The method of claim 1, wherein the biguanide antimicrobial agent is present in an amount ranging from 1 ppm to about 1 wt. % based upon the total amount the composition.

13. The method of claim 1, wherein the infectious disease is a fungal infection.

14. The method of claim 1, wherein the infectious disease is an amoeba infection.

15. The method of claim 14, wherein the amoeba infection is amoebal keratitis.

16. The method of claim 1, wherein the infectious disease is a viral infection.

17. The method of claim 1, wherein the infectious disease is a bacterial infection.

18. The method of claim 17, wherein the infectious disease is bacterial conjunctivitis.

19. A composition for treating infectious disease comprising an ointment base, and a biguanide antimicrobial agent in an amount effective to treat infectious disease.

20. The composition of claim 19, wherein the biguanide antimicrobial agent is selected from the group consisting of poly hexamethylene biguanide, chlorhexidine and Alexidine.

21. The composition of claim 19, wherein the ointment base is selected from the group consisting of petrolatum and mineral oil.

22. The composition of claim 19, wherein water, if any, is present in an amount that is a maximum of about 20 wt. %.

23. The composition of claim 19, further comprising water in an amount that is a minimum of about 20 wt. % and a maximum of about 60 wt. %.

24. The composition of claim 19, further comprising a surfactant.

25. The composition of claim 19, wherein the surfactant is selected from the group consisting of polysorbate, cremophor, triton, poloxamine, poloxamer and tyloxapol.

26. The composition of claim 19, wherein the surfactant is present in an amount ranging from about 1 wt. % to about 5 wt. %.

27. The composition of claim 19, wherein the composition further comprises a penetration enhancer.

28. The composition of claim 19, wherein the penetration enhancer is present in an amount that is a minimum of about 0.001 wt. % and a maximum of about 5 wt. %.

29. The composition of claim 19, wherein the biguanide antimicrobial agent is Alexidine.

30. The composition of claim 19, wherein the stabilizer is in an amount effective to extend the shelf life a minimum of about 10%.

31. The composition of claim 19, wherein the biguanide antimicrobial agent is present in an amount ranging from 1 ppm to about 0.1 wt. % based upon the total amount the composition.
